(19) United States
(12) Patent Application Publication
(10) Pub. No.: US 2018/0318142 A1
(43) Pub. Date: Nov. 8, 2018

(54) LOWER BODY SUPPORT AND COMPRESSION GARMENT

(71) Applicant: Monee Denine Gagliardo, Del Mar, CA (US)

(72) Inventor: Monee Denine Gagliardo, Del Mar, CA (US)

(21) Appl. No.: 15/970,885

(22) Filed: May 4, 2018

Related U.S. Application Data
(60) Provisional application No. 62/501,645, filed on May 4, 2017.

(51) Int. Cl. A61F 13/08; A41D 1/06; A41F 9/00; A41D 31/00; A61F 5/02

(52) U.S. Cl. CPC A61F 13/08; A41D 1/06; A61F 5/028; A41D 31/0005; A41F 9/00

(57) ABSTRACT

A fashion garment is constructed for use as comfortable outerwear from one or more stretchable materials shaped to fit snuggly over and provide support and compression to a wearer's lower body. The garment includes a waistband constructed to encircle and provide compression to the wearer's waist; a pair of leg panels coupled to each other and to the waistband and constructed to enwrap and provide compression to at least portions of the wearer's pelvis, buttocks, hips, and legs; a pair of hip panels coupled to the leg panels and positioned against the wearer's hips to provide additional compression to the hips; and a gusset panel coupled to the leg panels and positioned to cover the user's crotch area.

### CROSS-REFERENCE TO RELATED APPLICATION

This application claims priority from U.S. Provisional Application 62/501,645, filed May 4, 2017, and titled “Pelvis Support and Compression Garment.”

### FIELD OF THE DISCLOSURE

The present invention relates to a garment for support and compression of the lower portions of the human body, especially in and around the pelvic region.

### BACKGROUND

Patients who have undergone aesthetic or reconstructive surgery of the abdomen, genitals, or pelvis or who have given vaginal childbirth are likely candidates for some form of complications during the recovery period. Typical of such complications are atelectasis, hypostatic pneumonia, phlebitis, pulmonary complication, vulvar prolapse, abdominal swelling, low back and hip dysfunction, sacroiliac joint dysfunction, and lower back pain. Patients who have acute or chronic conditions such as cancer or severe arthritis or who have suffered injuries to their lower bodies often require support in the pelvic, hip, leg, or abdominal areas to promote healing and relief from pain.

There are many treatments to provide support for the lower body. Some techniques include exercise, manual manipulation such as chiropractic care, and cutaneous stimulation. Drugs may also be utilized, such as pain relievers and muscle relaxants. Invasive techniques including surgery are also used in extreme situations.

Some medical devices have been developed to address these problems. Hard immobile supports that interfere with mobility are common for people with chronic waist and lower back injuries or pain. Other devices provide support only to limited sections of the lower body, can be bulky, and often restrict mobility. Still others mechanically manipulate the body in ways that can be uncomfortable to the wearer. Special garments have also been developed to address these problems through structural support and compression of the wearer's pelvis and hips, but these garments tend to be uncomfortable to wear and often apply unwanted pressure in areas not needing it, to the end that the wearers are often eager to remove the garments to get relief from the discomfort the garment causes. In short, the devices and garments existing in the art tend to be bulky, unattractive, and uncomfortable, leaving the wearer feeling as though they are “necessary evils” in the quest for better health and relief from body pain.

### SUMMARY

Described here is a fashion garment constructed for use as comfortable outerwear from one or more stretchable materials shaped to fit snuggly over and provide support and compression to a wearer's lower body. The garment includes a waistband constructed to encircle and provide compression to the wearer's waist; a pair of leg panels coupled to each other and to the waistband and constructed to enwrap and provide compression to at least portions of the wearer's pelvis, buttocks, hips, and legs; a pair of hip panels coupled to the leg panels and positioned against the wearer's hips to provide additional compression to the hips; and a gusset panel coupled to the leg panels and positioned to cover the user's crotch area.

In some embodiments, the waistband includes at least two layers of material comprising two differing fabric blends, such as a polyester-spandex blend and a nylon-spandex blend. The waistband is also often constructed of sufficient length to cover and provide support for at least a portion of the lumbar region of the wearer's back.

For some embodiments the hip panels each includes one or more elastic bands constructed to extend over the hip in substantially parallel directions and along the wearer's leg to provide additional compression to the hips. The hip panels are also often constructed to have substantially triangular shape, with a base portion positioned nearer the wearer's waist and a tip portion positioned nearer the wearer's knee. The gusset panel is also often constructed to have substantially triangular shape.

For some embodiments of the garment, the one or more stretchable materials include materials having blends of approximately 88 percent polyester and 12 percent spandex and approximately 72 percent nylon and 28 percent spandex.

### BRIEF DESCRIPTION OF THE DRAWINGS

Embodiments of the invention are described below, by way of example, with reference to the accompanying drawings.

FIGS. 1A and 1B show front and rear views, respectively, of one embodiment of a lower body support and compression garment;

FIG. 2 shows a side view of the lower body support and compression garment of FIG. 1; and

FIG. 3 shows one example of a gusset panel for inclusion in a lower body support and compression garment like that of FIG. 1.

Like reference numerals refer to like parts throughout the drawings.

### DETAILED DESCRIPTION

Described here is a lower-body support garment designed to be a fashionable, form-fitting, and comfortable piece of everyday outerwear that supports the lower body in a way that promotes physiological health and relief from chronic pain. The garment typically takes the form of a pair of athletic or leisure-wear pants, such as those often used for activities like yoga and physical exercise, that extend from the wearer's waist or midriff over the hips and knees to the lower legs, providing support and compression to the wearer's pelvic region, hips, midriff, lower back, and buttocks, as described below. The support garment is comfortable, attractive, and ideal for wear during routine daily activities and exercise.

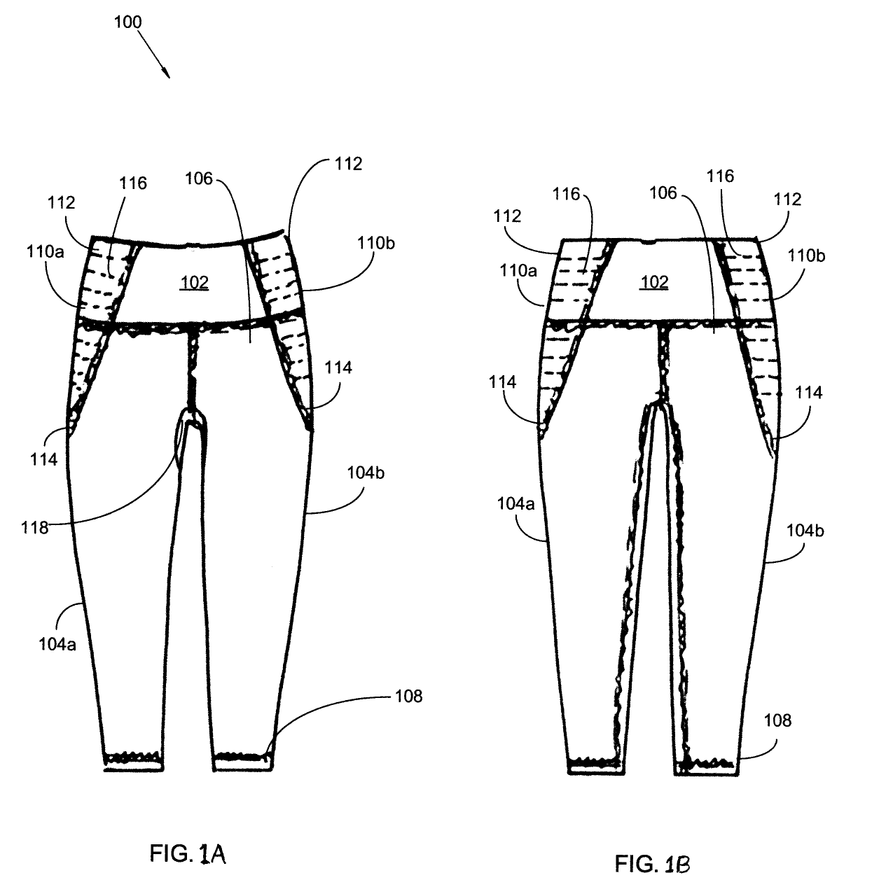

FIGS. 1A and 1B show a lower body support and compression garment 100 designed to extend from a wearer's waist downwardly to a point on the wearer's lower leg, below the knee. The garment 100 is manufactured from a soft, comfortable, light-weight, stretchable, breathable, and durable material, such as a nylon-spandex or polyester-spandex blend, that allows the garment to stretch over the form of the wearer's body and compress snugly against the wearer's skin. As shown here, the garment includes a waistband 102 of sufficient height to provide compression against and support for the wearer's waistline, midriff, and lumbar area of the spine. The waistband 102 in some embodiments also includes a reinforcing inner layer, such as one made from a soft, elastic, mesh-like material, that integrates into the waistband 102 for additional support in this region of the wearer's body. The waistband 102 can be worn in an upward position to provide support and compression over the wearer's midriff and lower back or, in the alternative, it can be worn in a downward position, doubled over the material that covers the buttocks and hips, to provide extra support below the waistline and to create a different fashionable aesthetic. Experimentation suggests that a material having a polyester-spandex blend in the ratio of approximately 88% polyester and 12% spandex is particularly well suited for use in the waistband 102, while material having a nylon-spandex blend of approximately 72% nylon and 28% spandex is well suited for use in the inner lining.

In certain embodiments the waistband 102 has a pair of free ends that are secured to each other through a fastener. Adjustments of pressure are made through hooks and eyes, ties and loops, buckles and straps, buttons, or other mechanisms that vary the relative positions of the ends of the waistband 102. In this manner, the wearer can adjust the amount of support and compression to the hip and midriff.

A pair of leg panels 104a-b attaches to and extends downward from the waistband 102, over the hips and pelvis, and around the legs and buttocks to provide support and compression to these portions of the wearer's body. The leg panels 104a-b also include an integral pair of hip panels 110a-b that align with the wearer's hips. The leg panels 104a-b and hip panels 110a-b are constructed from a resilient but comfortable elastic material (such as the 88/12 polyester-spandex blend described above) to provide multiple layers of structural support to each of the hips while also compressing the entire pelvic region into alignment. The hip panels 110a-b are particularly beneficial in urging the sacroiliac (SI) joints on each side of the wearer's body into proper position and alignment. The SI joints can be a particularly problematic part of the body, often causing chronic pain that is very difficult to remedy because of their unique physiological structure and deep internal positioning within the pelvic region. In many embodiments each of the hip panels 110a-b has a generally triangular shape, with the base of the triangle positioned nearer the wearer's waistline and the tip of the triangle pointing toward the wearer's knee.

One or more elastic support bands (not shown in FIGS. 1A and 1B) are integrated into each of the hip panels 110a-b, stretching in a latitudinal direction (along the length of the leg) to provide an additional source of compression against the hips. This latitudinal compression directly against the hips promotes proper alignment of the sacroiliac joint and is also beneficial for maintaining proper positioning of the hips during pregnancy. In many embodiments, each of the hip panels 110a-b includes multiple elastic bands running roughly parallel to and spaced apart from each other to distribute the compression forces evenly across the surface of the hip. Experimentation shows that four parallel elastic bands of roughly 4″ in width over each hip provide the ideal degree of support and compression. A material having the 88/12 polyester-spandex blend described above is well suited for construction of the elastic bands. A topstitch portion 116 is also incorporated into the each of the hip panels 110a-b to retain the elastic bands in a fixed position and, in some embodiments, to provide an attractive fashionable aesthetic.

The garment 100 also includes a gusset panel 118 positioned in the wearer's crotch area. The gusset panel 118 is constructed from a resilient, stretchable, moisture-wicking material (such as the 88/12 polyester-spandex blend) that inhibits moisture buildup and allows expansion of the garment in the crotch area to enhance comfort during physical activity. In many embodiments the gusset has a generally triangular shape, with the tip of the triangle pointing toward the rear of the wearer's body.

The garment 100 as shown in FIGS. 1A and 1B takes the form of a long pair of form-fitting pants. In other embodiments the garment 100 takes the form of knee-length (bicycle style) pants, short pants, “boy short” style pants, and brief pants (“briefs”). The garment 100 is fabricated from a generally stretchable, resilient material that provides support to the lower body and compresses the pelvic region and hips. The resilient material may include, for example, a blend of nylon, polyester, and spandex and may have the appearance of yoga style pants. In some embodiments, the degree of support and compression to the hips and lower body is adjustable through use of a fastener at the waistband 102 or through donning of smaller or larger sized garments. Additionally, the garment 100 has the advantage of being comfortable, supportive, and aesthetically pleasing as a fashionable item of outerwear. Because of its form-fitting construction, the garment 100 eliminates the need for underwear and thus is not subject to unsightly “panty lines.”

Those skilled in the art will recognize that many sports injuries develop from overuse of the hips. Most hip pain is from stress on the joint, and inflammation can be caused when the femoral head doesn't fit properly in the acetabulum or has reduced cartilage. This is a medical condition commonly found in athletes or as a result of the normal aging process. It is also known that hip problems often occur during pregnancy. In pregnancy, most hip pain is referred from the sciatic nerve, lateral femoral cutaneous nerve inflammation, and even an inguinal hernia. Further, bursitis known as Trochanteric Bursitis can occur in the joint from inflammation due to weight gain in pregnancy and from overuse in athletes.

In addition, sprains (injury to ligaments in the joint) and strains (muscle or tendon damage) are common during pregnancy and through injuries in sports. The iliotibial (IT) band of muscles may also become inflamed and cause Piriformis Syndrome, most commonly in runners.

The support and compression garment 100 described here is useful in combatting all of these problems. The garment 100 may be donned during pregnancy to provide relief and healing of hip injuries due to the weight of the carried child and by athletes who repetitively run and jump cause injury to the hip and pelvis region. The garment 100 is especially effective in supporting and compressing the hips to allow for stabilization of the sacroiliac joints and to provide support and comfort to the lower body during activities or during recovery.

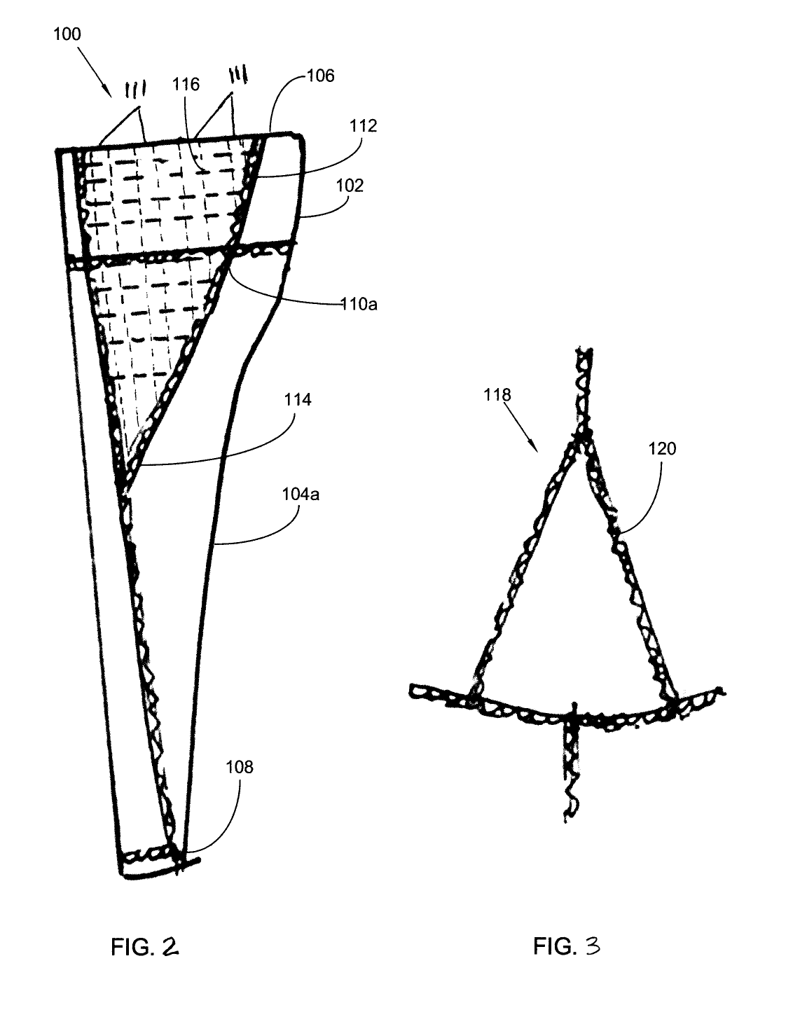

FIG. 2 shows the garment 100 in side view and illustrates clearly how the hip panel 110a aligns along the corresponding hip. The hip panels 110a-b in some embodiments (as shown here) extend along the full height of the waistband 102 and over the leg panels 104a-b. The size and shape of the hip panels 110a-b will vary, as desired by the garment designer, to accommodate different genders, sizes, and even pregnancy growth of the wearers.

In some embodiments, a plurality of elastic support bands 111 integrate into the hip panels 110a-b. As described above, the elastic bands 111 are configured to compress the hips and are disposed in a lateral, spaced-apart configuration, such that all the bands to stretch in a generally latitudinal direction (along the length of the leg). This lateral compression works to support and align the pelvis and particularly the sacroiliac joints. The elastic bands 111 are represented in the figure by dashed lines because they are integrated into the hip panels 110a-b and are not visible to the observer.

Thus, the elastic support bands 111 reinforce the material of the hip panels 110a-b, which in turn reinforce the material of the leg panels 104a-b, to compress the hips and the surrounding pelvis region for proper alignment of the sacroiliac joint. In this manner, hip and pelvis problems, such as sacroiliac joint dysfunction, are at least partially treated and remedied.

The topstitch portion 116 also integrates into the hip panels 110a-b, running in the longitudinal direction (parallel to the wearer's waist), roughly perpendicular to the elastic bands 111. In one embodiment, the topstitch portion 116 is visible on the outer surface of the hip panels 110a-b and serves as an element of the garment's fashion design. The topstitch portion 116 helps secure the elastic bands 111 in place along the hip panels 110a-b. As shown here, the topstitch portion 116 comprises four rows of topstitch, shown as dashed lines that represent the stitching.

Referring now to FIG. 3, the gusset panel 118 is positioned in alignment with the crotch area for purposes of inhibiting moisture buildup, strengthening the garment in the crotch area, and increasing the stretch capacity of the garment 100 for enhanced comfort in the crotch area. A gusset stitching 120 is typically used to couple the gusset panel 118 to the leg panels 104a-b. The gusset panel 118 is typically defined by a generally triangular shape.

Many modifications, variations, and changes in detail can be made to the embodiments described above without departing from the spirit and scope of the invention as defined in the claims that follow. It is intended therefore that all items in the foregoing description and shown in the accompanying drawings be interpreted as illustrative and not limiting.

What is claimed is:

1. A fashion garment constructed for use as comfortable outerwear from one or more stretchable materials shaped to fit snuggly over and provide support and compression to a wearer's lower body, the garment comprising:
a waistband constructed to encircle and provide compression to the wearer's waist;
a pair of leg panels coupled to each other and to the waistband and constructed to enwrap and provide compression to at least portions of the wearer's pelvis, buttocks, hips, and legs;
a pair of hip panels coupled to the leg panels and positioned against the wearer's hips to provide additional compression to the hips; and
a gusset panel coupled to the leg panels and positioned to cover the user's crotch area.

2. The garment of claim 1, where the waistband comprises at least two layers of material comprising two differing fabric blends.

3. The garment of claim 2, where at least one of the layers comprises a polyester-spandex blend and at least one other of the layers comprises a nylon-spandex blend.

4. The garment of claim 1, where the waist panel is constructed of sufficient length to cover and provide support for at least a portion of the lumbar region of the wearer's back.

5. The garment of claim 1, where the hip panels each includes one or more elastic bands constructed to extend over the hip and along the wearer's leg to provide additional compression to the hips.

6. The garment of claim 4, where the hip panels each includes multiple elastic bands extending over the wearer's hip in substantially parallel directions.

7. The garment of claim 1, where each of the hip panels is constructed to have substantially triangular shape, with a base portion positioned nearer the wearer's waist and a tip portion positioned nearer the wearer's knee.

8. The garment of claim 1, where the gusset panel is constructed to have substantially triangular shape.

9. The garment of claim 1, where the one or more stretchable materials include a material having a blend of approximately 88 percent polyester and 12 percent spandex.

10. The garment of claim 1, where the one or more stretchable materials include a material having a blend of approximately 72 percent nylon and 28 percent spandex.
